(19) United States
(12) Patent Application Publication
(10) Pub. No.: US 2006/0134463 A1
(43) Pub. Date: Jun. 22, 2006

(54) Triarylamine-based compound for organic electroluminescent device and organic electroluminescent device employing the same

(76) Inventors: Seok-Hwan Hwang, Suwon-si (KR); Young-Kook Kim, Suwon-si (KR); Chang-Ho Lee, Suwon-si (KR); Seok-Jong Lee, Suwon-si (KR); Seung-Gak Yang, Suwon-si (KR); Hee-Yeon Kim, Suwon-si (KR)

Correspondence Address: Robert E. Bushnell, Suite 300, 1522 K Street, N.W., Washington, DC, 20005, US

(21) Appl. No.: 11/302,447

(22) Filed: Dec. 14, 2005

(30) Foreign Application Priority Data
Dec. 20, 2004 (KR) 10-2004-0108825

(51) Int. Cl. H01L 51/54; H05B 33/14; C07C 211/00; C07C 209/06

(52) U.S. Cl. 428/690; 564/434; 564/405; 428/917; 313/504; 313/506; 257/E51.051

(57) ABSTRACT

A triarylamine-based compound represented by Formula (1), and an organic electroluminescent device using an organic layer including the triarylamine-based compound are provided: 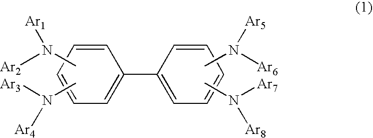
where each of Ar₁ to Ar₈ is independently a substituted or unsubstituted C6-C30 aryl group or a substituted or unsubstituted C2-C30 heteroaryl group. The triarylamine-based compound has superior electric properties and charge transport abilities, and thus is useful as a hole injection material and a hole transport material which are suitable for fluorescent and phosphorescent devices of all colors, including red, green, blue, and white colors. The organic electroluminescent device manufactured using the triarylamine-based compound has high efficiency, low voltage, high luminance, and a long lifespan.

### CROSS-REFERENCE TO RELATED PATENT APPLICATION AND CLAIM OF PRIORITY

This application claims the benefit of Korean Patent Application No. 10-2004-0108825, filed on Dec. 20, 2004, in the Korean Intellectual Property Office, the disclosure of which is incorporated herein in its entirety by reference.

### BACKGROUND OF THE INVENTION

1. Field of the Invention

The present invention relates to a triarylamine-based compound for an organic electroluminescent device and an organic electroluminescent device employing the same, and more particularly, to a triarylamine-based compound which has electric stability, superior charge transport ability and high glass transition temperature and can prevent crystallization, and an organic electroluminescent device using an organic layer including the same.

2. Description of the Related Art

An electroluminescent (EL) device which is a self-emission type display device has received significant attention owing to its merits of a wide viewing angle, superior contrast, and rapid response. An EL device is divided into an inorganic EL device in which an emitting layer is composed of an inorganic compound, and an organic EL device in which an emitting layer is composed of an organic compound. An organic EL device has superior luminance, driving voltage, and response rate to an inorganic EL device and can display multicolors, and thus much research into organic EL devices has been conducted.

The organic EL device generally has a layered structure of anode/organic emitting layer/cathode. When a hole transport layer and/or a electron injection layer is further interposed between the anode and the emitting layer or between the emitting layer and the cathode, an anode/hole transport layer/organic emitting layer/cathode structure or an anode/hole transport layer/organic emitting layer/electron injection layer/cathode structure is formed.

The hole transport layer is known to be composed of a triphenylamine derivative or an anthracene derivative (see, for example, U.S. Pat. Nos. 6,646,164 and 6,465,115).

Organic EL devices including hole transport layers composed of conventional materials are not satisfactory in terms of lifespan, efficiency and power consumption, and thus a material for a hole transport layer with a significant improvement in such characteristics is required.

### SUMMARY OF THE INVENTION

The present invention provides a hole transport and/or hole injection layer material which has electric stability, superior charge transport ability, and high glass transition temperature, can prevent crystallization, and is suitable for fluorescent and phosphorescent devices having all colors, including red, green, blue, and white colors, etc., and a method of preparing the same.

The present invention also provides an organic EL device using an organic layer composed of the above-described material and having high efficiency, low voltage, high luminance and long lifespan.

According to an aspect of the present invention, there is provided a triarylamine-based compound represented by Formula (1) for an organic electroluminescent device: 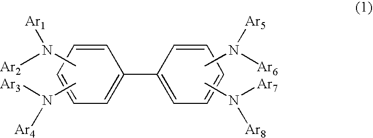
where each of Ar₁ to Ar₈ is independently a substituted or unsubstituted C6-C30 aryl group or a substituted or unsubstituted C2-C30 heteroaryl group.

According to another aspect of the present invention, there is provided a method of preparing a triarylamine-based compound represented by Formula (3) for an organic electroluminescent device by reacting a compound (A) with diarylamine (B) to obtain the compound represented by Formula (3): 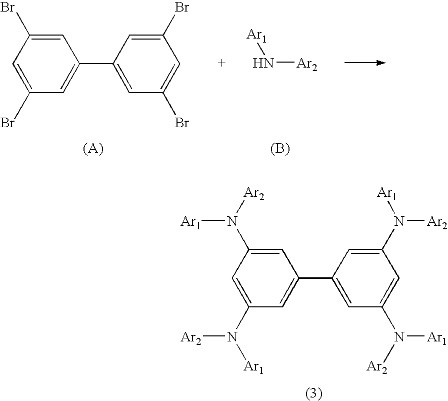
where each of Ar₁ and Ar₂ is independently a substituted or unsubstituted C6-C30 aryl group or a substituted or unsubstituted C2-C30 heteroaryl group.

The reaction is carried out in the presence of Pd₂(dba)₃ (dba=dibenzylideneacetone), sodium tert-butoxide, and tri(tert-butyl) phosphine at 50 to 150° C.

The compound (A) may be obtained by reacting 1,3,5-tribromobenzene with butyllithium, and then reacting the resulting product with copper chloride at −78 to 0° C.:

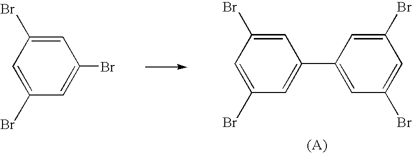

According to another aspect of the present invention, there is provided an organic EL device including a first electrode, a second electrode, and an organic layer interposed therebetween, in which the organic layer contains the triarylamine-based compound.

### BRIEF DESCRIPTION OF THE DRAWINGS

A more complete appreciation of the present invention, and many of the above and other features and advantages of the present invention, will be readily apparent as the same becomes better understood by reference to the following detailed description when considered in conjunction with the accompanying drawings in which like reference symbols indicate the same or similar components, wherein:

FIG. 1 is a cross-sectional view of an organic EL device according to an embodiment of the present invention;

FIG. 2 illustrates a UV spectrum of Compound 3 according to an embodiment of the present invention;

FIG. 3 illustrate Thermo Gravimetric Analysis (TGA) results of Compound 3 obtained according to an embodiment of the present invention;

FIG. 4 illustrate Differential Scanning Calorimetry (DSC) results of Compound 3 obtained according to an embodiment of the present invention;

FIG. 5 illustrates a UV spectrum of Compound 6 according to an embodiment of the present invention;

FIG. 6 illustrates a UV spectrum of Compound 39 according to an embodiment of the present invention;

FIG. 7 illustrates a UV spectrum of Compound 41 according to an embodiment of the present invention;

FIG. 8 illustrates a UV spectrum of Compound 50 according to an embodiment of the present invention;

FIG. 9 is a graph illustrating the variation in luminance with respect to voltage in organic EL devices obtained in Examples 1-3 according to embodiments of the present invention and Comparative Example 1;

FIG. 10 is a graph illustrating the variation in current efficiency with respect to the luminance in organic EL devices obtained in Examples 1-3 according to embodiments of the present invention and Comparative Example 1;

FIG. 11 is a graph illustrating the variation in luminous intensity with respect to time in organic EL devices obtained in Example 1 according to an embodiment of the present invention and Comparative Example 1;

FIG. 12 is a graph illustrating the variation in current density with respect to voltage in organic EL devices obtained in Example 3 according to an embodiment of the present invention and Comparative Example 2; and

FIG. 13 is a graph illustrating the variation in luminance with respect to voltage in organic EL devices obtained in Example 3 according to an embodiment of the present invention and Comparative Example 2.

### DETAILED DESCRIPTION OF THE INVENTION

Hereinafter, the present invention will be described in more detail.

The present invention provides a compound represented by Formula (1), which has four triarylamine derivatives in its molecule, a method of preparing the same, and an organic EL device using a material for an organic layer such as a hole injection layer, a hole transport layer, or an emitting layer: 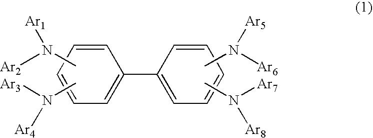
where each of Ar₁ to Ar₈ is independently a substituted or unsubstituted C6-C30 aryl group or a substituted or unsubstituted C2-C30 heteroaryl group.

Preferably, in Formula (1), Ar₁ to Ar₈ are a phenyl group, a methylphenyl group, a dimethyl group, a trimethyl group, an ethylphenyl group, an ethylbiphenyl group, an o-, m-, or p-fluorophenyl group, a dichlorophenyl group, a dicyanophenyl group, a trifluoromethoxyphenyl group, an o-, m-, or p-tolyl group, an o-, m-, or p-cumenyl group, a mecityl group, a phenoxyphenyl group, a (α,α-dimethylbenzene)phenyl group, a (N,N′-dimethyl)aminophenyl group, a (N,N′-diphenyl)aminophenyl group, a pentarenyl group, an indenyl group, a naphthyl group, a methylnaphthyl group, an anthracenyl group, an azurenyl group, a heptarenyl group, an acenaphthylrenyl group, a fluorenyl group, an anthraquinolyl group, a methylanthryl group, a phenanthrenyl group, a triphenylene group, a pyrenyl group, a crycenyl group, an ethyl-crycenyl group, a pycenyl group, a pherylenyl group, a chloropherylenyl group, a pentaphenyl group, a pentacenyl group, a tetraphenylenyl group, a hexaphenyl group, a hexacenyl group, a rubicenyl group, a coroneryl group, a trinaphthylenyl group, a heptaphenyl group, a heptacenyl group, a pyranthrenyl group, an ovarenyl group, a carbazolyl group, a lower alkylcarbazolyl group, a biphenyl group, a lower alkyl phenyl group, a lower alkoxy phenyl group, a thiophenyl group, an indolyl group, or a pyridyl group. The lower alkyl group may be a C1-C5 alkyl and the lower alkoxy group may be a C1-C5 alkoxy.

More preferably, Ar₁ to Ar₈ may be an aryl group having 1-3 rings selected from a phenyl group and a naphthyl group, or an aryl group substituted with 1-3 groups among a C1-C3 lower alkoxy group, a cyano group, a phenoxy group, a phenyl group or a halogen atom.

In Formula (1), Ar₁ to Ar₈ may be substituted with a C1-C10 alkyl group, a C1-C10 alkoxy group, a nitro group, a halogen atom, an amino group, a C6-C10 aryl group, a C2-C10 heteroaryl group, a cyano group, or a hydroxy group.

The triarylamine-based compound used in the present invention has high glass transition point or melting point due to four bulky triarylamine groups having steric hindrance. Thus, a resistance to joule heat generated in organic layers including the triarylamine-based compound, between such organic layers, or between such an organic layer and a metal electrode, and a resistance to high temperature conditions increase when electroluminescence occurs, and therefore, when the triarylamine-based compound is used as a hole injection layer, a hole transport layer, or an emitting material or a host material of an emitting layer of an organic EL device, it exhibits high luminance and can emit light for a long time. In particular, since crystallization is prevented due to four bulky triarylamine groups in a molecule, said effects can be further increased.

An organic EL device of the present invention has high durability when it is stored and operated. This is because the compound used in the present invention has four triarylamine derivatives, and thus has a high glass transition temperature Tg.

The compound represented by Formula (1) may be a compound represented by Formula (2):

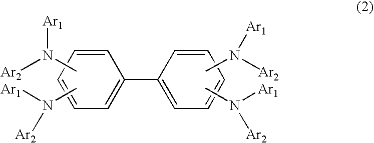

where Ar₁ is a p-tolyl group, a 4-cyanophenyl group, or a naphthyl group; and Ar₂ is a p-tolyl group, a phenyl group, or a 4-methoxyphenyl group.

The compound represented by Formula (2) may be a compound represented by Formula (3): 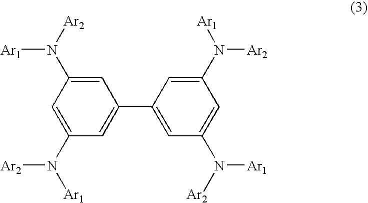
where Ar₁ is a p-tolyl group, a 4-cyanophenyl group, or a naphthyl group; and Ar₂ is a p-tolyl group, a phenyl group, or a 4-methoxyphenyl group.

Examples of the triarylamine-based compound represented by Formula (1) are provided, but are not limited thereto:

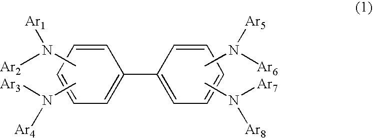

where A1 through A8 may be the groups described in Table 1 and Table 2:

TABLE 1
R = Ar₁ = Ar₂ = Ar₃ = Ar₄ = Ar₅ = Ar₆ = Ar₇ = Ar₈

| No. | R |
|---|---|
| 1 | —C₂H₅ |
| 2 | 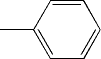 |
| 3 | 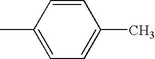 |
| 4 | 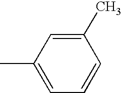 |
| 5 | 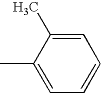 |
| 6 | 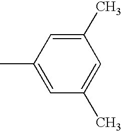 |
| 7 | 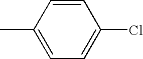 |
| 8 | 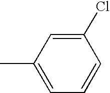 |
| 9 | 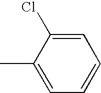 |
| 10 | 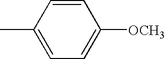 |
| 11 | 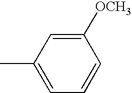 |
| 12 | 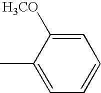 |
| 13 | 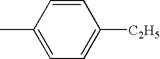 |
| 14 | 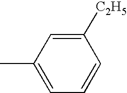 |
| 12 | 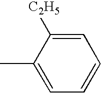 |
| 13 | 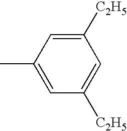 |
| 14 | 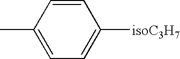 |
| 15 | 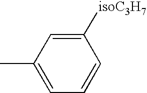 |
| 16 | 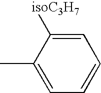 |
| 17 | 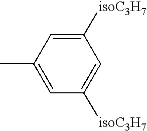 |
| 18 | 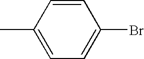 |
| 19 | 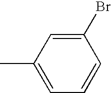 |
| 20 | 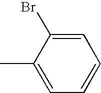 |
| 21 | 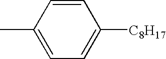 |
| 22 | 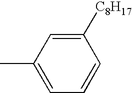 |
| 23 | 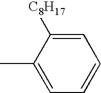 |
| 24 | 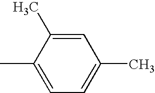 |
| 25 | 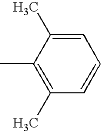 |
| 26 | 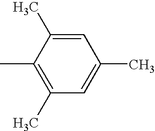 |
| 27 | 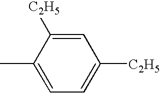 |
| 28 | 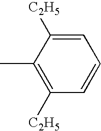 |
| 29 | 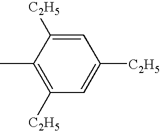 |
| 30 | 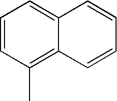 |
| 31 | 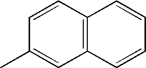 |
| 32 | 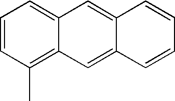 |
| 33 | 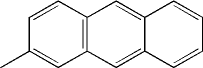 |

and

TABLE 2
R1 = Ar₁ = Ar₂ = Ar₃ = Ar₄, R2 = Ar₅ = Ar₆ = Ar₇ = Ar₈

| No. | R1 | R2 |
|---|---|---|
| 34 | —C₂H₅ | 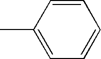 |
| 35 | 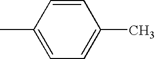 | 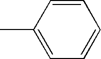 |
| 36 | 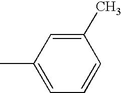 | 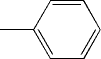 |
| 37 | 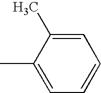 | 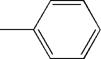 |
| 38 | 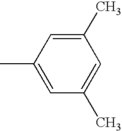 | 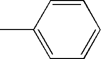 |
| 39 | 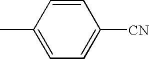 | 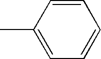 |
| 40 | 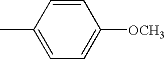 | 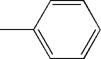 |
| 41 | 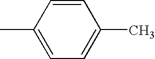 | 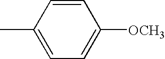 |
| 42 | 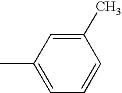 | 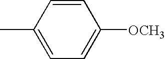 |
| 43 | 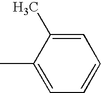 | 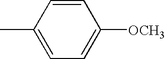 |
| 44 | 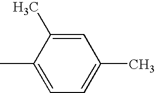 | 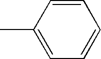 |
| 45 | 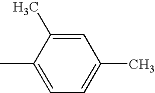 | 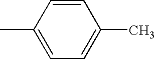 |
| 46 | 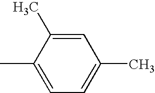 | 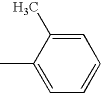 |
| 47 | 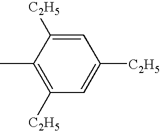 | 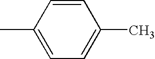 |
| 48 | 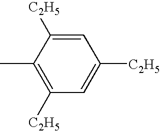 | 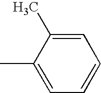 |
| 49 | 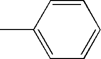 | 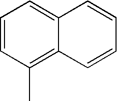 |
| 50 | 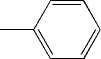 | 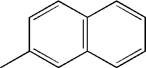 |
| 51 | 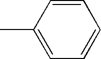 | 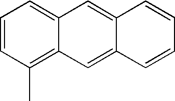 |
| 52 | 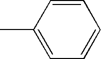 | 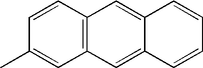 |
| 53 | 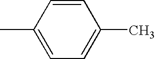 | 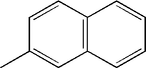 |
| 54 | 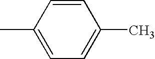 | 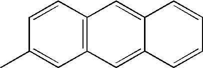 |
| 55 | 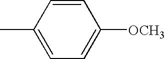 | 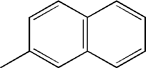 |
| 56 | 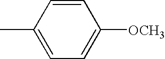 | 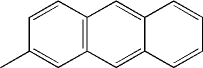 |
| 57 | 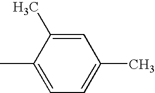 | 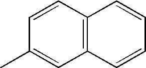 |

A synthesis method of the triarylamine-based compound represented by Formula (1) will now be described. Among the compounds of Formula (1), the synthesis method of the compound represented by Formula (3) is exemplified. The compound represented by Formula (3) is synthesized by the following method.

First, 1,3,5-tribromobenzene is reacted with butyllithium. The resulting product is reacted with CuCl₂ to obtain Compound (A). In the coupling reaction, a reaction temperature is −78 to 0° C., and preferably about −78° C.

The Compound (A) is reacted with diarylamine (B) to obtain the compound represented by Formula (3). In this reaction, bases such as Pd₂(dba)₃ (dba=dibenzylideneacetone) and sodium tert-butoxide, and tri(tert-butyl) phosphine (P(t-Bu)3) are used, and a reaction temperature is in the range of 50-150° C.

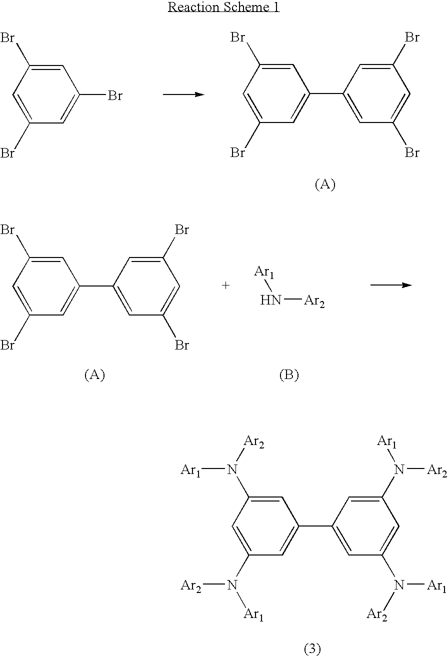

An organic EL device of the present invention will now be described. An organic EL device of the present invention includes an organic layer having the triarylamine-based compound represented by Formula (1). The organic layer containing the triarylamine-based compound represented by Formula (1) may be a hole injection layer or a hole transport layer, or a single layer serving as both a hole injection layer and a hole transport layer.

When the organic layer is a hole injection layer or a hole transport layer, the organic EL device may have a first electrode/hole injection layer/emitting layer/second electrode structure, a first electrode/hole injection layer/emitting layer/hole transport layer/second electrode structure, or a first electrode/emitting layer/hole transport layer/second electrode structure.

The emitting layer is composed of a phosphorescent or fluorescent material.

In the organic EL device according to an embodiment of the present invention, the organic layer containing the triarylamine-based compound represented by Formula (1) may be an emitting layer.

When the emitting layer is formed of the triarylamine-based compound, the triarylamine-based compound is used as a fluorescent or phosphorescent host.

A method of manufacturing an organic EL device according to an embodiment of the present invention will now be described.

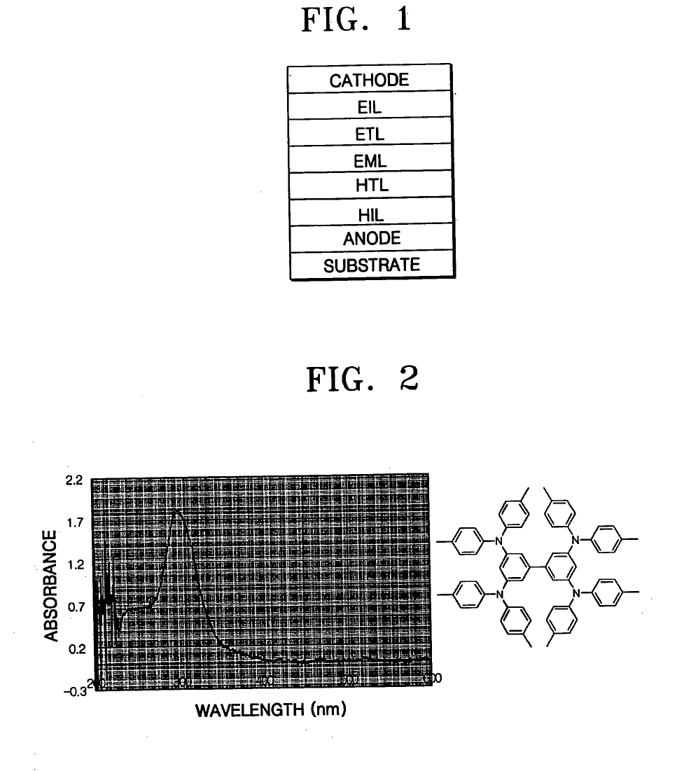

FIG. 1 is a cross-sectional view of an organic EL device according to an embodiment of the present invention.

First, a material for forming anode, which has a high work function, is deposited or sputtered on a substrate to form an anode. Any substrate used in a conventional organic EL device is used, and a glass substrate or a transparent plastic substrate having good mechanical strength, thermal stability, transparency, surface softness, manipulability, and water-proofness may be used. The anode may be composed of indium tin oxide (ITO), indium zinc oxide (IZO), tin oxide (SnO₂), or zinc oxide (ZnO), which is transparent and have good conductivity.

Next, a hole injection layer (HIL) is formed on the anode using a vacuum evaporation, spin coating, casting, or Langmuir-Blodgett (LB) method. The HIL may be formed using the vacuum evaporation method, since it is easy to obtain uniform film quality and the generation of pinholes is suppressed.

When the HIL is formed using the vacuum evaporation method, the deposition conditions can be varied according to the type of compound used as a hole injection material, and the desired structure and thermal property of the HIL, but may include a deposition temperature of 50 to 500° C., a vacuum of 10⁻⁸ to 10⁻³ torr, a deposition rate of 0.01 to 100 Å/sec, and a film thickness of 10 Å to 5 μm.

A HIL material is not particularly restricted, but may be the compound represented by Formula (1), a phthalocyanine-based compound, such as CuPc, as disclosed in U.S. Pat. No. 4,356,429 which is incorporated herein by reference, or a Starburst type amine derivatives, for example, TCTA, m-MTDATA, or m-MTDAPB, as described in Advanced Material, 6, p. 677 (1994), which is incorporated herein by reference.

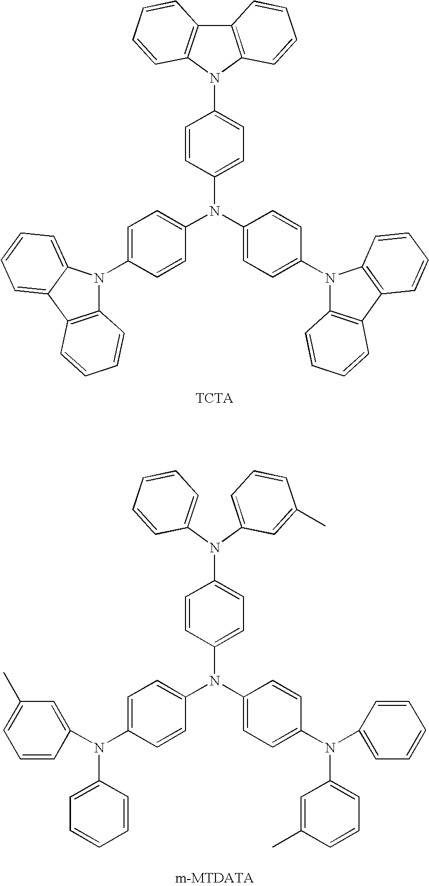

Then, a hole transport layer (HTL) is formed on the HIL using a vacuum evaporation, spin coating, casting, or LB method. The HTL may be formed using the vacuum evaporation method since it is easy to obtain uniform film quality and the generation of pinholes is suppressed. When the HTL is formed using the vacuum evaporation method, the deposition conditions vary according to the type of compound to be used, but may be almost identical to the deposition conditions for the HIL.

A HTL material is not particularly restricted, but may be the triarylamine-based compound represented by Formula (1), or any compound known to be used in the HTL. For example, carbazole derivatives, such as N-phenylcarbazole and polyvinylcarbazole, general amine derivatives having an aromatic condensed ring, such as N,N′-bis(3-methylphenyl)-N,N′-diphenyl-[1,1-biphenyl]-4,4′-diamine (TPD), N,N′-di(naphthalen-1-yl)-N,N′-diphenyl benzidine (NPD), etc. are used.

Then, an emitting layer (EML) is formed on the HTL using a vacuum evaporation, spin coating, casting, or LB method. The EML may be formed using the vacuum evaporation method since it is easy to obtain uniform film quality and the generation of pinholes is suppressed. When the EML is formed using the vacuum evaporation method, the deposition conditions vary according to the type of compound to be used, but may be almost identical to the deposition conditions for the HIL.

An EML material is not particularly restricted, but may be the triarylamine-based compound represented by Formula (1) as a fluorescent or phosphorescent host. Alq₃ (3-phenyl-4-(1′-naphthyl)-5-phenyl-1,2,4-triazole (TAZ), s-TAZ, tris(8-quinolinolato)-aluminum) may be used as a fluorescent host. IDE102 and IDE105 available from Idemitsu Kosan Co., Ltd., and C545T available from Hayashibara may be used as fluorescent dopants, and Ir(PPY)₃ (PPy=phenylpyridine) (green), F2Irpic (bis[2-(4,6-difluorophenyl)pyridinato-N,C2′] iridium picolinate) (blue), and RD61 (red) available from UDC may be vacuum evaporated (doped) in combination as phosphorescent dopants.

The concentration of the dopant is not particularly restricted, but is typically 0.01-15 parts by weight based of total 100 parts by weight of the host and the dopant.

When the phosphorescent dopant is used in the EML, a hole blocking material is vacuum evaporated or spin coated on the EML to form a hole blocking layer (HBL) (not shown), in order to prevent a triplet exciton or a hole from diffusing into the ETL. The hole blocking material is not particularly restricted, but may be any material which is used as a hole blocking material in the art. For example, oxadiazole derivatives or triazole derivatives, phenanthroline derivatives, or hole blocking materials described in JP 11-329734 (A1) which is incorporated herein by reference and the like may be used and, more particularly, BAlq (bis(2-methyl-8-quinolinolato)(4-phenylphenolato)aluminum) and BCP (2,9-dimethyl-4,7-diphenyl-1,10-phenanthroline) are used.

Then, an electron transport layer (ETL) is formed using a vacuum evaporating, spin coating, or casting method. Preferably, the ETL is formed using the vacuum evaporating method. An ETL material may be any material which can stably transport electrons injected from an electron injection electrode (cathode) and, more particularly, tris(8-quinolinolate) aluminum (Alq₃) may be used. In addition, an electron injection layer (EIL) for facilitating the injection of electrons from the cathode may be deposited on the ETL and a material therefor is not particularly restricted.

LiF, NaCl, CsF, Li₂O, BaO, etc. may be used as the EIL material. The deposition conditions of the HBL, the ETL, and the EIL vary according to the type of compound to be used, but may be almost identical to the deposition conditions for HIL.

Finally, a metal for a cathode is vacuum evaporated or sputtered on the EIL to form a cathode. The metal for a cathode may be a metal having a low work function, alloy, electric conducting compound, and a mixture thereof. Examples of such a material include Li, Mg, Al, Al—Li, Ca, Mg—In, or Mg—Ag. Also, a transmittance type cathode composed of ITO or IZO may form a front surface of a light emitting device.

The organic EL device according to an embodiment of the present invention may include one or two intermediate layers in addition to the anode, the HIL, the HTL, the EML, the ETL, the EIL, and the cathode as illustrated in FIG. 1.

The compound represented by Formula (1) which is a light emitting material having superior light emitting property and hole transport property, is useful as a hole injection material and/or a hole transport material of blue, green, red fluorescent and phosphorescent devices, and may also be used as a host material.

Synthesis Examples of organic light emitting materials having four triarylamine derivatives as branched chains i.e., Compound 3, Compound 6, Compound 39, Compound 41, and Compound 50, and Examples of the organic EL device including the organic light emitting materials will be described in greater detail. (Compound 3 is the compound represented by Formula (1), wherein A₁ to A₈ are the group of No. 3 in the above Table, Compound 6 is the compound represented by Formula (1), wherein A₁ to A₈ are the groups of No. 6 in the above Table, Compound 39 is the compound represented by Formula (1), wherein A₁ to A₈ are the groups of No. 39 in the above Table, Compound 41 is the compound represented by Formula (1), wherein A₁ to A₈ are the groups of No. 41 in the above Table, and Compound 50 is the compound represented by Formula (1), wherein A₁ to A₈ are the groups of No. 50 in the above Table), The following examples are for illustrative purposes and are not intended to limit the scope of the invention.

### SYNTHESIS EXAMPLE

### Preparation of Compound 3

Compound 3 was synthesized according to the reaction scheme 2.

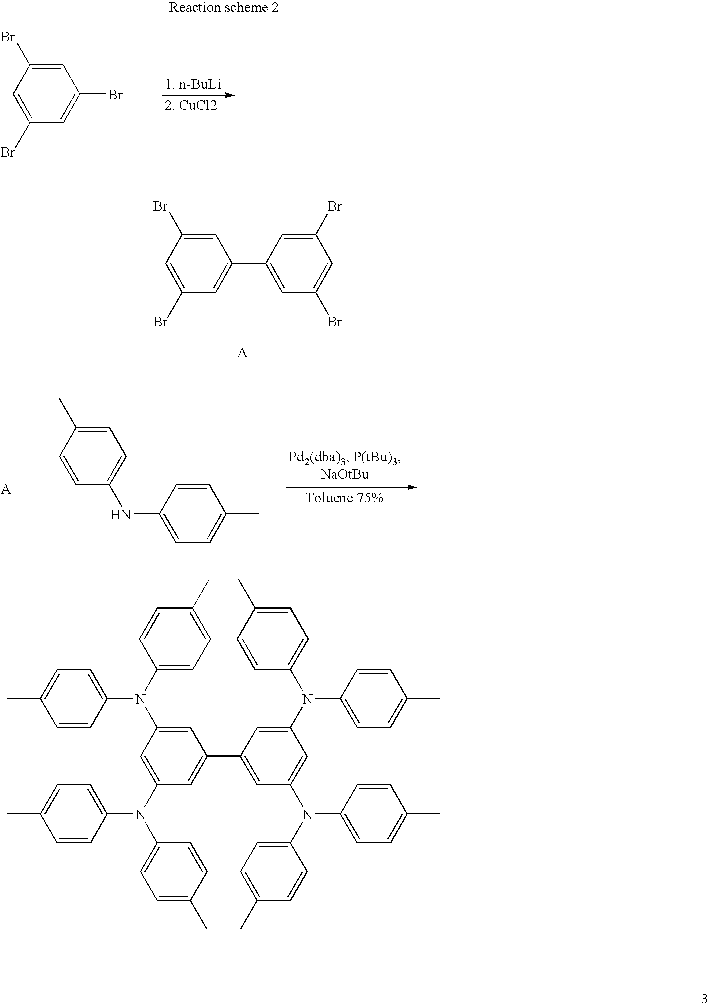

### Synthesis of Intermediate Compound A

6.3 g (20 mmol) of 1,3,5-tribromobenzene was dissolved in 50 mL of diethyl ether, and then cooled to −78° C. Then, 8.8 ml (22 mmol, 2.5 M in hexane) of n-butyllithium was slowly added to the solution. After stirring at −78° C. for 1 hour, 2.96 g (22 mmol) of CuCl₂ was added. After stirring for 5 hours, the mixture was washed with distilled water and ethyl acetate at room temperature. The washed ethyl acetate layer was dried on MgSO₄, and then dried under reduced pressure to obtain a crude product. The crude product was purified with a silica gel column chromatography to obtain 3.74 g of the Intermediate compound A as a white solid (yield: 80%).

¹H NMR (CDCl₃, 400 MHz) δ (ppm) 7.69 (s, 2H), 7.58 (s, 4H); ¹³C NMR (CDCl₃, 100 MHz) δ (ppm) 141.7, 133.8, 128.9, 123.5.

### Synthesis of Compound 3

246 mg (0.5 mmol) of the intermediate compound A, 600 mg (3 mmol) of di-p-tolylamine, 500 mg (5 mmol) of t-BuONa, 40 mg (0.04 mmol) of Pd₂(dba)₃, and 10 mg (0.04 mmol) of P(t-Bu)₃ were dissolved in 5 mL of toluene, and then stirred at 90° C. for 3 hours. The reaction mixture was cooled to room temperature and three times extracted with 20 mL of distilled water and diethyl ether. The collected organic layer was dried on magnesium sulfate and the solvent was evaporated. The residue was purified with a silica gel column chromatography to obtain 545 mg of Compound 3 as a white solid (yield: 75%).

¹H NMR (CDCl₃, 300 MHz) δ (ppm) 6.92 (dd, 32H), 6.56 (s, 6H), 2.28 (s, 24H); ¹³C NMR (CDCl₃, 100 MHz) δ (ppm) 148.6, 145.0, 142.8, 131.9, 129.6, 124.2, 116.5, 115.2, 20.8.

The obtained Compound 3 was diluted with CHCl₃ to a concentration of 0.2 mM and the UV spectrum therefor was obtained. A maximum absorption wavelength of 299 nm was observed in the UV spectrum (FIG. 2).

Further, Compound 3 was subjected to thermal analysis using TGA (Thermo Gravimetric Analysis) and DSC (Differential Scanning Calorimetry) (N₂ atmosphere, temperature range: room temperature—600° C. (10° C./min)—TGA, room temperature—400° C.—DSC, Pan type: Pt pan in disposable Al pan (TGA), disposable Al pan (DSC)).

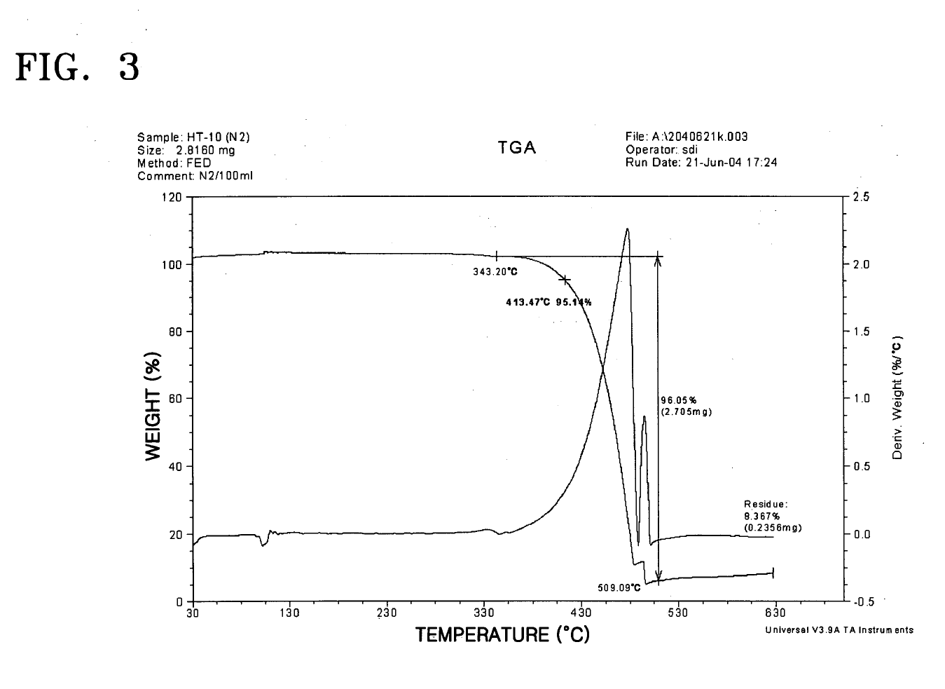

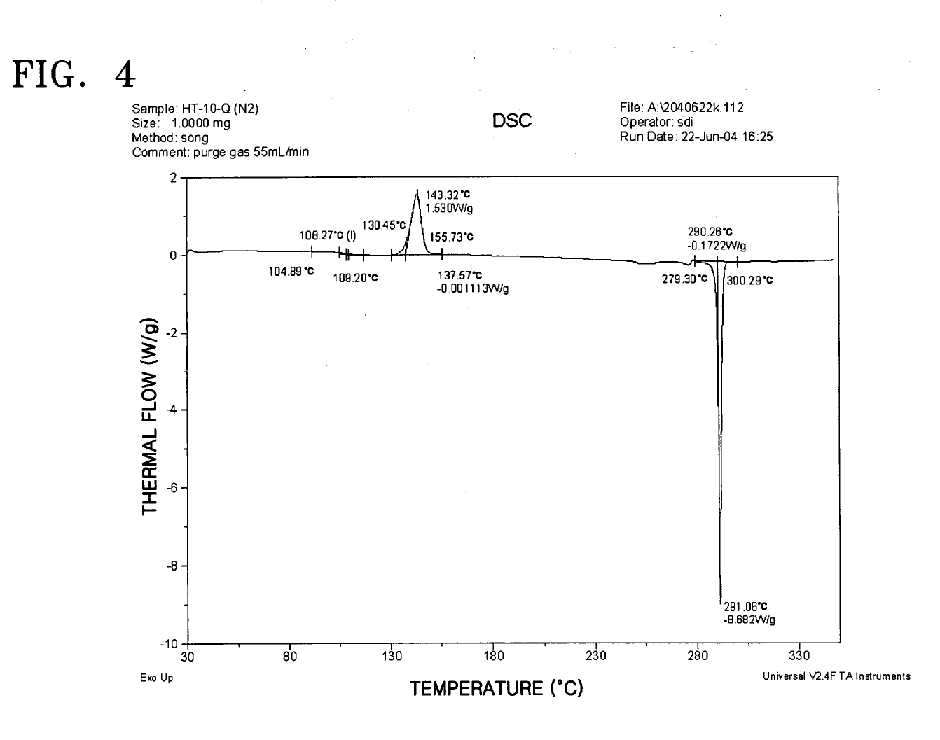

As a result, Td 414° C., Tg 108° C., Tc 173° C., and Tm 291° C. were obtained (FIGS. 3 and 4).

A HOMO (Highest Occupied Molecular Orbital) energy level of 5.30 eV and a LUMO (Lowest Occupied Molecular Orbital) energy level of 2.23 eV were obtained using UV absorption spectrum and a potentiometer AC-2.

### SYNTHESIS EXAMPLE 2

### Preparation of Compound 6

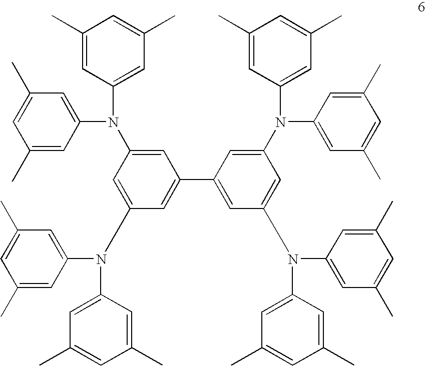

Compound 6 was synthesized in the same manner as Synthesis Example 1 using intermediate compound A and bis-3,5-dimethylbenzeneamine (yield 75%).

¹H NMR (C₃D₃, 300 MHz) δ (ppm) 7.20 (d, 4H), 7.09 (t, 2H), 6.84 (s, 16H), 6.45 (s, 8H), 2.01 (s, 48H); ¹³C NMR (C₃D₃, 100 MHz) δ (ppm) 150.1, 148.3, 143.8, 138.8, 125.2, 123.2, 117.6, 116.4, 21.2.

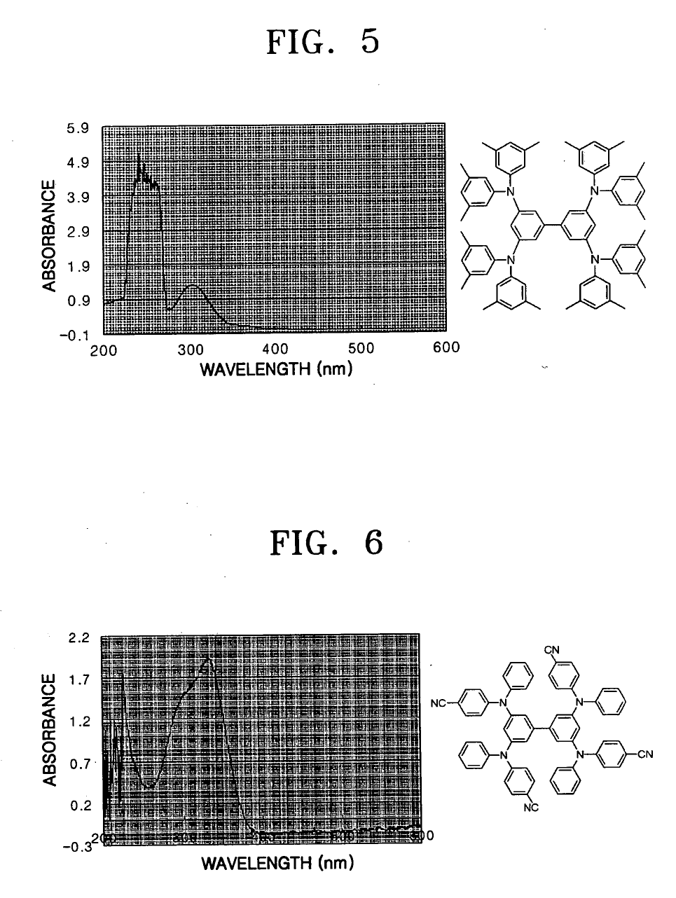

The obtained Compound 6 was diluted with CHCl₃ to a concentration of 0.2 mM and the UV spectrum therefor was obtained. A maximum absorption wavelength of 303 nm was observed in the UV spectrum (FIG. 5).

Further, Compound 6 was subjected to thermal analysis using TGA (Thermo Gravimetric Analysis) and DSC (Differential Scanning Calorimetry) (N₂ atmosphere, temperature range: room temperature—600° C. (10° C./min)—TGA, room temperature—400° C.—DSC, Pan type: Pt pan in disposable Al pan (TGA), disposable Al pan (DSC)).

As a result, Td 387° C., Tg 165° C., and Tm 289° C. were obtained.

A HOMO (Highest Occupied Molecular Orbital) energy level of 5.25 eV and a LUMO (Lowest Occupied Molecular Orbital) energy level of 2.18 eV were obtained using UV absorption spectrum and a potentiometer AC-2.

### SYNTHESIS EXAMPLE 3

### Preparation of Compound 39

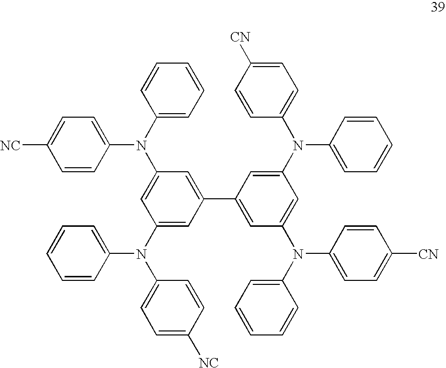

Compound 39 was synthesized in the same manner as Synthesis Example 1 using the intermediate compound A and 4-phenylaminobenzonitrile (yield 80%).

¹H NMR (CDCl₃, 300 MHz) δ (ppm) 7.40-7.28 (m, 16H), 7.21 (td, 4H), 7.08 (d, 8H), 6.94 (d, 8H), 6.79 (t, 2H), 6.72 (d, 4H); ¹³C NMR (CDCl₃, 100 MHz) δ (ppm) 151.2, 148.1, 145.8, 143.2, 133.5, 130.2, 126.6, 125.9, 121.3, 120.9, 119.8, 119.6, 103.9.

The obtained Compound 39 was diluted with CHCl₃ to a concentration of 0.2 mM and the UV spectrum therefor was obtained. A maximum absorption wavelength of 332 nm was observed in the UV spectrum (FIG. 6).

Further, Compound 39 was subjected to thermal analysis using TGA (Thermo Gravimetric Analysis) and DSC (Differential Scanning Calorimetry) (N₂ atmosphere, temperature range: room temperature—600° C. (10° C./min)—TGA, room temperature—400° C.—DSC, Pan type: Pt pan in disposable Al pan (TGA), disposable Al pan (DSC)).

As a result, Td 468° C., Tg 136° C., and Tm 186° C. were obtained.

A HOMO (Highest Occupied Molecular Orbital) energy level of 5.55 eV and a LUMO (Lowest Occupied Molecular Orbital) energy level of 2.40 eV were obtained using UV absorption spectrum and a potentiometer AC-2.

### SYNTHESIS EXAMPLE 4

### Preparation of Compound 41

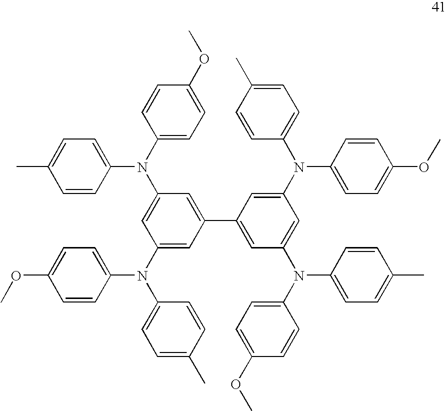

Compound 41 was synthesized in the same manner as Synthesis Example 1 using the intermediate compound A and 4-methoxyphenyl-p-tolylamine (yield 92%).

¹H NMR (CD₂Cl₂, 300 MHz) δ (ppm) 7.10-6.95 (m, 18H), 6.88 (d, 8H), 6.77 (d, 6H), 6.45 (dd, 6H), 3.78 (s, 12H), 2.29 (s, 12H); ¹³C NMR (C₃D₃, 100 MHz) δ (ppm) 156.4, 149.4, 145.5, 143.2, 140.9, 132.3, 129.9, 127.2, 124.0, 114.8, 114.4, 113.6, 55.7, 20.8.

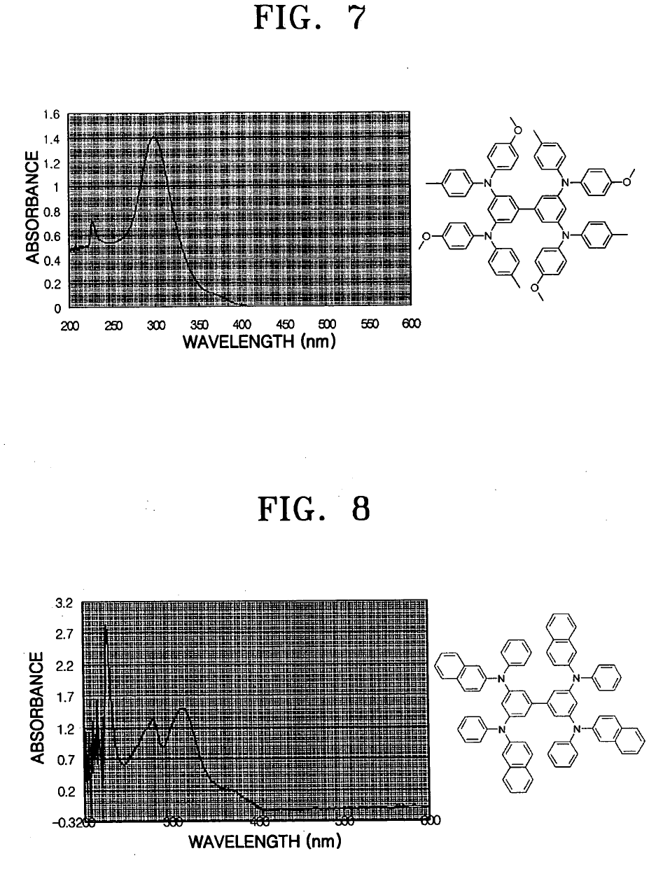

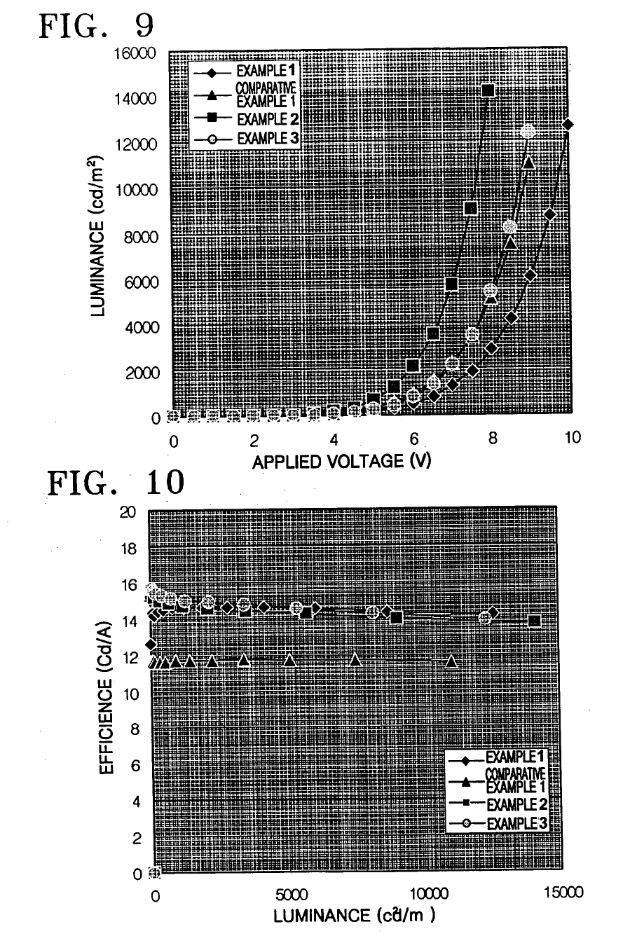

The obtained Compound 41 was diluted with CHCl₃ to a concentration of 0.2 mM and the UV spectrum therefor was obtained. A maximum absorption wavelength of 299 nm was observed in the UV spectrum (FIG. 7).

Further, Compound 41 was subjected to thermal analysis using TGA (Thermo Gravimetric Analysis) and DSC (Differential Scanning Calorimetry) (N₂ atmosphere, temperature range: room temperature—600° C. (10° C./min)—TGA, room temperature—400° C.—DSC, Pan type: Pt pan in disposable Al pan (TGA), disposable Al pan (DSC)).

As a result, Td 408° C., Tg 100° C., Tc 153° C., and Tm 234° C. were obtained.

A HOMO (Highest Occupied Molecular Orbital) energy level of 5.30 eV and a LUMO (Lowest Occupied Molecular Orbital) energy level of 2.26 eV were obtained using UV absorption spectrum and a potentiometer AC-2.

### SYNTHESIS EXAMPLE 5

### Preparation of Compound 50

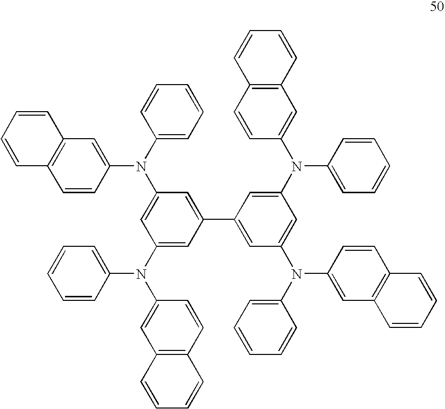

Compound 50 was synthesized in the same manner as Synthesis Example 1 using the intermediate compound A and 2-naphthylphenylamine (yield 91%).

¹H NMR (CDCl₃, 300 MHz) δ (ppm) 7.67 (dd, 4H), 7.53 (t, 8H), 7.37-7.29 (m, 12H), 7.20 (dd, 4H), 7.10-7.03 (m, 16H), 6.92-6.85 (m, 4H), 6.80 (t, 2H), 6.69 (d, 4H), 7.03 (d, 4H); ¹³C NMR (CDCl₃, 100 MHz) δ (ppm) 148.6, 147.2, 144.9, 142.9, 134.3, 130.0, 129.1, 128.7, 127.5, 126.9, 126.1, 124.4, 123.0, 120.4, 118.0, 116.7.

The obtained Compound 50 was diluted with CHCl₃ to a concentration of 0.2 mM and the UV spectrum therefor was obtained. A maximum absorption wavelength of 315 nm was observed in the UV spectrum (FIG. 8).

Further, Compound 50 was subjected to thermal analysis using TGA (Thermo Gravimetric Analysis) and DSC (Differential Scanning Calorimetry) (N₂ atmosphere, temperature range: room temperature—600° C. (10° C./min)—TGA, room temperature—400° C.—DSC, Pan type: Pt pan in disposable Al pan (TGA), disposable Al pan (DSC)).

As a result, Td 504° C., Tg 118° C., and Tm 259° C. were obtained.

A HOMO (Highest Occupied Molecular Orbital) energy level of 5.30 eV and a LUMO (Lowest Occupied Molecular Orbital) energy level of 2.23 eV were obtained using UV absorption spectrum and a potentiometer AC-2.

### EXAMPLE 1

A corning 15 Ω/cm² (1200 Å) ITO glass substrate as an anode was cut to a size of 50 mm×50 mm×0.7 mm and ultrasonically washed with isopropyl alcohol and pure water, for 5 min each wash. Then, the washed glass substrate was irradiated with a UV radiation for 30 min and washed by exposing to ozone, and then, installed in a vacuum evaporator. Compound 6 was vacuum evaporated on the substrate to form a 600 Å thick HIL.

Then, 4,4′-bis[N-(1-naphthyl)-N-phenylamino]biphenyl (NPB) was vacuum evaporated on the HIL to form a 300 Å thick HTL. Alq₃, which was known as a green fluorescent host, and C545T, which was known as a green fluorescent dopant, were co-deposited (weight ratio 98:2) on the HTL to form a 200 Å thick EML.

Alq₃ was deposited on the EML to form a 300 Å thick ETL, and then LiF was deposited on the ETL to form a 10 Å thick EIL and Al was deposited thereon to form a 3000 Å thick cathode, thereby completing the organic electroluminescent device as illustrated FIG. 1.

This device has a current density of 3.38 mA/cm², a luminance of 490.3 cd/m², a color coordination (0.30, 0.64), and luminous efficiency of 14.49 cd/A at a driving voltage of 6.0 V.

### Comparative Example 1

An organic EL device was manufactured in the same manner as in Example 1, except that IDE 406 (available from Idemitsu Kosan Co., Ltd.) instead of for an organic electroluminescent device was used to form a HIL.

This device has a current density of 7.76 mA/cm², a luminance of 905.5 cd/m², a color coordination (0.30, 0.64), and luminous efficiency of 11.56 cd/A at a driving voltage of 6.0 V.

### EXAMPLE 2

A corning 15 Ω/cm² (1200 Å) ITO glass substrate as an anode was cut to a size of 50 mm×50 mm×0.7 mm and ultrasonically washed with isopropyl alcohol and pure water, for 5 min each wash. Then, the washed glass substrate was irradiated with a UV radiation for 30 min and washed by exposing to ozone, and then, installed in a vacuum evaporator. Compound 6 was vacuum evaporated on the substrate to form a 600 Å thick hole injection and hole transport layer.

Alq₃, which was known as a green fluorescent host, and C545T, which was known as a green fluorescent dopant, were co-deposited (weight ratio 98:2) on the hole injection and hole transport layer to form a 200 Å thick EML. Alq₃ was deposited on the EML to form a 300 Å thick ETL, and then LiF was deposited on the ETL to form a 10 Å thick EIL and Al was deposited thereon to form a 3000 Å thick cathode, thereby completing an organic electroluminescent device.

This device has a current density of 14.41 mA/cm², a luminance of 2,094 cd/m², a color coordination (0.31, 0.64), and luminous efficiency of 14.55 cd/A at a driving voltage of 6.0 V.

### EXAMPLE 3

An organic EL device was manufactured in the same manner as in Example 1, except that the thickness of the hole injection and hole transport layer of Compound 6 was 800 Å.

This device has a current density of 5.10 mA/cm², a luminance of 762.7 cd/m², a color coordination (0.31, 0.64), and luminous efficiency of 15.12 cd/A at a driving voltage of 6.0 V.

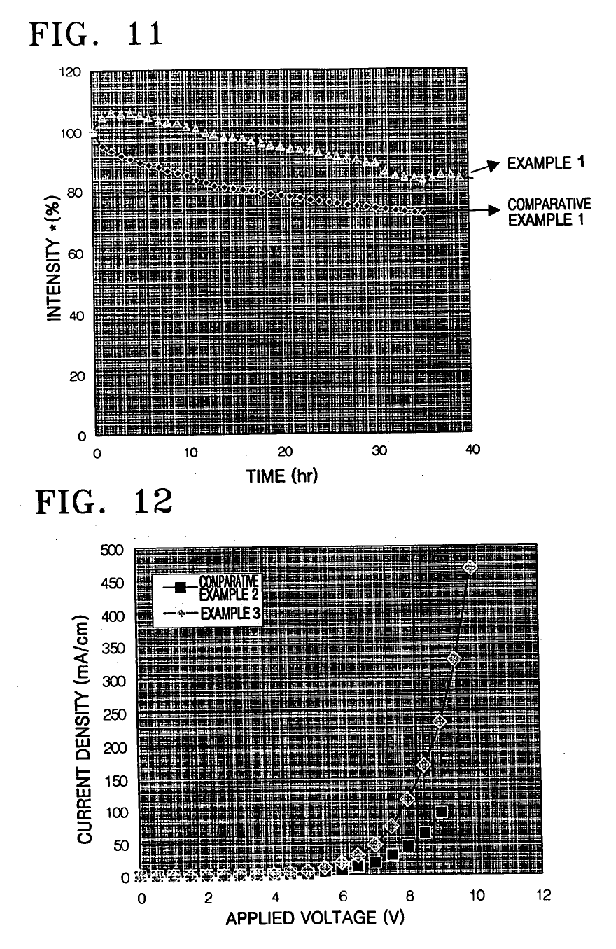

The organic EL device using Compound 6 of the present invention for the HIL has a good hole injection ability close to the organic EL device of Comparative Example 1 due to an increased current efficiency although density and driving voltage were somewhat reduced. Further, lifespan characteristic was improved although driving voltage was somewhat increased at the same luminance (FIG. 11).

The driving voltage of the organic EL device which used Compound 6 according to an embodiment of the present invention for the hole injection and hole transport layer was approximately 1 V lower than the driving voltage of the organic EL device of Comparative Example 1 due to an improved charge injection ability. Further, the organic EL device of the present invention had higher current density, current efficiency and luminance than the organic EL device of Comparative_Example 1.

### EXAMPLE 4

A corning 15 Ω/cm² (1200 Å) ITO glass substrate as an anode was cut to a size of 50 mm×50 mm×0.7 mm and ultrasonically washed with isopropyl alcohol and pure water, for 5 min each wash. Then, the washed glass substrate was irradiated with a UV radiation for 30 min and washed by exposing to ozone, and then, installed in a vacuum evaporator. IDE406 was vacuum evaporated on the substrate to form a 600 Å thick HIL.

Then, Compound 6 was vacuum evaporated on the HIL to form a 300 Å thick HTL. Alq₃, which was a green fluorescent host and C545T, which was a green fluorescent dopant, were co-deposited (weight ratio 98:2) on the HTL to form a 200 Å EML.

Alq₃ was deposited on the EML to form a 300 Å thick ETL, and then LiF was deposited on the ETL to form a 10 Å thick EIL and Al was deposited thereon to form a 3000 Å thick cathode, thereby completing an organic EL device.

This device has a current density of 18.0 mA/cm², a luminance of 2,076 cd/m², a color coordination (0.31, 0.64), and luminous efficiency of 11.54 cd/A at a driving voltage of 6.0 V.

### Comparative Example 2

An organic EL device was manufactured in the same manner as in Example 4, except that NPB instead of Compound 6 was used to form a HTL.

This device has a current density of 7.76 mA/cm², a luminance of 905.5 cd/m², a color coordination (0.30, 0.64), and luminous efficiency of 11.56 cd/A at a driving voltage of 6.0 V.

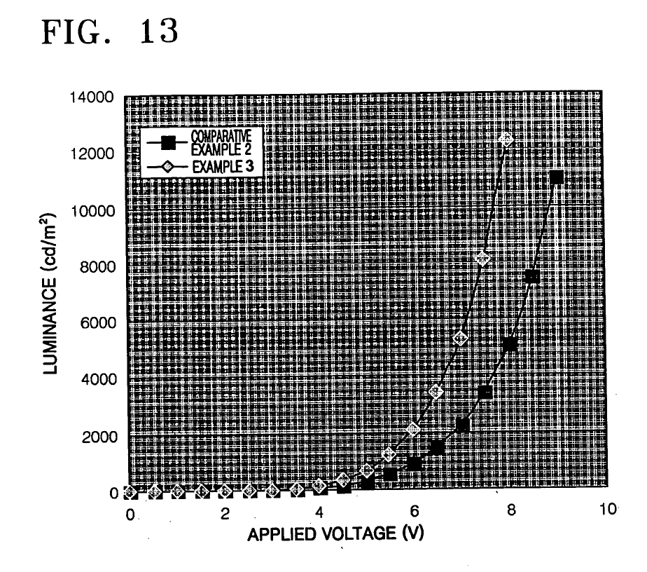

The driving voltage of the organic EL device in which Compound 6 was used for the HIL was approximately 1 V lower than the driving voltage of the organic EL device of Comparative Example 2 due to an improved charge injection ability. Further, the current density of the organic EL device of the present invention was significantly improved and the luminance of the organic EL device of the present invention was 2 times or more the luminance of the organic EL device of Comparative Example 2. FIGS. 12 and 13 illustrate variation in current density and luminance with respect to voltage.

As described above, the triarylamine-based compound according to an embodiment of the present invention has superior electric properties and charge transport abilities, and thus is useful as a hole injection material and a hole transport material which are suitable for fluorescent and phosphorescent devices of all colors, including red, green, blue, and white colors. The organic EL device manufactured using the triarylamie-based compound has high efficiency, low voltage, high luminance, and a long lifespan.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A compound represented by Formula (1): 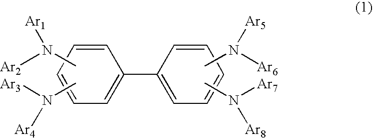
where each of Ar₁ to Ar₈ is independently a substituted or unsubstituted C6-C30 aryl group or a substituted or unsubstituted C2-C30 heteroaryl group.

2. The compound of claim 1, wherein each of Ar₁ to Ar₈ is independently a phenyl group, an methylphenyl group, a dimethyl group, a trimethyl group, an ethylphenyl group, an ethylbiphenyl group, an o-, m-, or p-fluorophenyl group, a dichlorophenyl group, a dicyanophenyl group, a trifluoromethoxyphenyl group, an o-, m-, or p-tolyl group, an o-, m-, or p-cumenyl group, a mecityl group, a phenoxyphenyl group, a (α,α-dimethylbenzene)phenyl group, a (N,N′-dimethyl)aminophenyl group, a (N,N′-diphenyl)aminophenyl group, a pentarenyl group, an indenyl group, a naphthyl group, a methylnaphthyl group, an anthracenyl group, an azurenyl group, a heptarenyl group, an acenaphthylrenyl group, a phenarenyl group, a fluorenyl group, an anthraquinolyl group, a methylanthryl group, a phenanthreyl group, a triphenylene group, a pyrenyl group, a crycenyl group, an ethyl-crycenyl group, a pycenyl group, a pherylenyl group, a chloropherylenyl group, a pentaphenyl group, a pentacenyl group, a tetraphenylenyl group, a hexaphenyl group, a hexacenyl group, a rubicenyl group, a coroneryl group, a trinaphthylenyl group, a heptaphenyl group, a heptacenyl group, a pyranthrenyl group, an ovarenyl group, a carbazolyl group, a lower alkyl carbazolyl group, a biphenyl group, a lower alkyl biphenyl group, a lower alkoxybiphenyl group, a thiophenyl group, an indolyl group, or a pyridyl group.

3. The compound of claim 1, which is a compound represented by Formula (2): 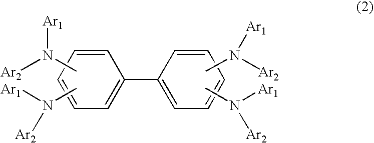
where each of Ar₁ to Ar₈ is independently a substituted or unsubstituted C6-C30 aryl group or a substituted or unsubstituted C2-C30 heteroaryl group.

4. The compound of claim 1, which is

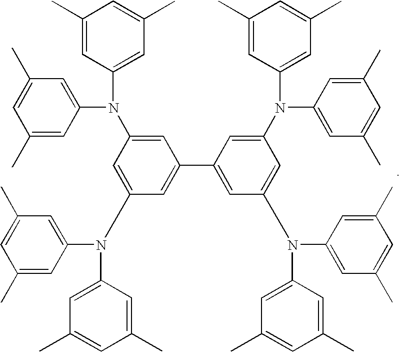

5. A method of preparing a compound represented by Formula (3), the method comprising:
reacting a compound (A) with diarylamine (B): 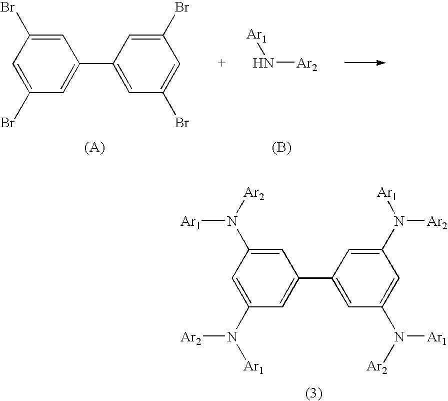
where each of Ar₁ and Ar₂ is independently a substituted or unsubstituted C6-C30 aryl group or a substituted or unsubstituted C2-C30 heteroaryl group.

6. The method of claim 5, wherein the reaction is carried out in the presence of Pd₂(dba)₃ (dba=dibenzylideneacetone), sodium tert-butoxide, and tri(tert-butyl)phosphine at a temperature of 50-150° C.

7. The method of claim 5, wherein the compound (A) is prepared by reacting 1,3,5-tribromobenzene with butyllithium, and then reacting the resulting product with copper chloride.

8. The method of claim 7, wherein the reaction is carried out at a temperature of −78 to 0° C.

9. The compound prepared by the method of claim 5.

10. An organic electroluminescent display device, comprising:
a first electrode;
a second electrode; and
an organic layer interposed between the first electrode and the second electrode, the organic layer comprising the compound represented by Formula (1): 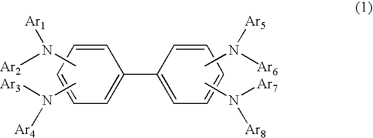
where each of Ar₁ to Ar₈ is independently a substituted or unsubstituted C6-C30 aryl group or a substituted or unsubstituted C2-C30 heteroaryl group.

11. The organic electroluminescent device of claim 10, wherein the organic layer is a hole injection layer or a hole transport layer.

12. The organic electroluminescent device of claim 10, wherein the organic layer is a single layer serving as both a hole injection layer and a hole transport layer.

13. The organic electroluminescent device of claim 10, wherein the organic layer is an emitting layer.

14. The organic electroluminescent device of claim 13, wherein the compound represented by Formula (1) is used as a fluorescent or phosphorescent host in the emitting layer.

15. The organic electroluminescent device of claim 10, wherein the organic layer is at least one of a hole injection layer, a hole transport layer, a single layer serving as both a hole injection layer and a hole transport layer, and an emitting layer.

16. The organic electroluminescent device of claim 10, wherein each of Ar₁ to Ar₈ is independently a phenyl group, an methylphenyl group, a dimethyl group, a trimethyl group, an ethylphenyl group, an ethylbiphenyl group, an o-, m-, or p-fluorophenyl group, a dichlorophenyl group, a dicyanophenyl group, a trifluoromethoxyphenyl group, an o-, m-, or p-tolyl group, an o-, m-, or p-cumenyl group, a mecityl group, a phenoxyphenyl group, a (α,α-dimethylbenzene)phenyl group, a (N,N′-dimethyl)aminophenyl group, a (N,N′-diphenyl)aminophenyl group, a pentarenyl group, an indenyl group, a naphthyl group, a methylnaphthyl group, an anthracenyl group, an azurenyl group, a heptarenyl group, an acenaphthylrenyl group, a phenarenyl group, a fluorenyl group, an anthraquinolyl group, a methylanthryl group, a phenanthreyl group, a triphenylene group, a pyrenyl group, a crycenyl group, an ethyl-crycenyl group, a pycenyl group, a pherylenyl group, a chloropherylenyl group, a pentaphenyl group, a pentacenyl group, a tetraphenylenyl group, a hexaphenyl group, a hexacenyl group, a rubicenyl group, a coroneryl group, a trinaphthylenyl group, a heptaphenyl group, a heptacenyl group, a pyranthrenyl group, an ovarenyl group, a carbazolyl group, a lower alkyl carbazolyl group, a biphenyl group, a lower alkyl biphenyl group, a lower alkoxybiphenyl group, a thiophenyl group, an indolyl group, or a pyridyl group.

17. The organic electroluminescent device of claim 10, wherein the compound is represented by Formula (2): 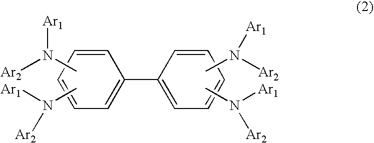
where each of Ar₁ to Ar₈ is independently a substituted or unsubstituted C6-C30 aryl group or a substituted or unsubstituted C2-C30 heteroaryl group.

18. The organic electroluminescent device of claim 9, wherein the compound is represented by the formula: [chemical structure]
